(19) United States
(12) Patent Application Publication
(10) Pub. No.: US 2008/0086137 A1
(43) Pub. Date: Apr. 10, 2008

(54) Fixation of femoral neck fractures

(76) Inventor: Robert Probe, Temple, TX (US)

Correspondence Address: TROP PRUNER & HU, PC, 1616 S. VOSS ROAD, SUITE 750, HOUSTON, TX, 77057-2631, US

(21) Appl. No.: 11/897,511

(22) Filed: Aug. 30, 2007

Related U.S. Application Data
(60) Provisional application No. 60/828,172, filed on Oct. 4, 2006.

(51) Int. Cl. A61B 17/58

(52) U.S. Cl. 606/69; 606/96

(57) ABSTRACT

In one embodiment of the invention, a device for femoral neck fracture fixation includes a base, a first conduit that slidably receives a first screw, a second conduit that slidably receives a second screw, and a third conduit that slidably receives a third screw. The first conduit is spaced from the second conduit to allow the first screw to couple to the femoral neck inferior cortex and to further allow the second screw and third screw to each couple to the femoral head and reduce angular instability of the femoral neck fracture.

This application claims priority to U.S. Provisional Patent Application No. 60/828,172 filed on Oct. 4, 2006 in the name of Robert Probe, entitled FIXATION OF FEMORAL NECK FRACTURES.

### BACKGROUND

With the aging of the world population, the incidence of fractures of the femoral neck is expected to increase dramatically. These fractures are typically treated with either internal fixation or prosthetic replacement. The most common form of internal fixation chosen by orthopaedic surgeons is the use of multiple cannulated screws. This surgical procedure offers advantages of simplicity and minimal invasiveness. Unfortunately, because of inadequate stability provided by these implants, the failure rate of this procedure remains high. This complication rate associated with cannulated screws has caused many physicians to explore alternative implants for internal fixation. The sliding hip screw is one of these alternatives. While initially designed for intertrochanteric fractures, some surgeons are attracted by the fact that this implant maintains a constant angular relationship between the screw, which crosses the neck fracture, and the femoral shaft. Despite these potential advantages, the torque required for insertion of the sliding hip screw can be deleterious while the angular stability provided is not optimal. Because neither of these existing fixation methods is ideal, many surgeons are resorting to removal of the femoral head and prosthetic replacement. This group of procedures requires a much larger surgical insult with associated increased short-term mortality. It is our belief that an implant which provides improved angular control and axial stability would reduce the number of complications currently seen with internal fixation. This, in turn, may reduce the need for hip replacement in the setting of femoral neck fracture.

### BRIEF DESCRIPTION OF THE DRAWINGS

The accompanying drawings, incorporated in and constituting a part of this specification, illustrate one or more implementations consistent with the principles of the invention and, together with the description of the invention, explain such implementations. The drawings are not necessarily to scale, the emphasis instead being placed upon illustrating the principles of the invention. In the drawings:

FIG. 1 includes a front view of a device in one embodiment of the invention.

FIG. 2 includes a side view of a device in one embodiment of the invention.

FIG. 3 includes a side view of a device in one embodiment of the invention.

FIG. 4 includes a side view of a device in one embodiment of the invention.

FIG. 5 includes a method in one embodiment of the invention.

### DETAILED DESCRIPTION

The following description refers to the accompanying drawings. Among the various drawings the same reference numbers may be used to identify the same or similar elements. While the following description provides a thorough understanding of the various aspects of the claimed invention by setting forth specific details such as particular structures, architectures, interfaces, and techniques, such details are provided for purposes of explanation and should not be viewed as limiting. Moreover, those of skill in the art will, in light of the present disclosure, appreciate that various aspects of the invention claimed may be practiced in other examples or implementations that depart from these specific details. At certain junctures in the following disclosure, descriptions of well known devices and methods have been omitted to avoid clouding the description of the present invention with unnecessary detail. Furthermore, in the following discussion and in the claims, the terms “including” and “comprising” are used in an open-ended fashion, and thus should be interpreted to mean “including, but not limited to . . . ”. Also, the term “couple” or “couples” is intended to mean either an indirect or direct mechanical, electrical, or other communicative connection. Thus, if a first component couples to a second component, that connection may be through a direct connection, or through an indirect connection via other devices and connections.

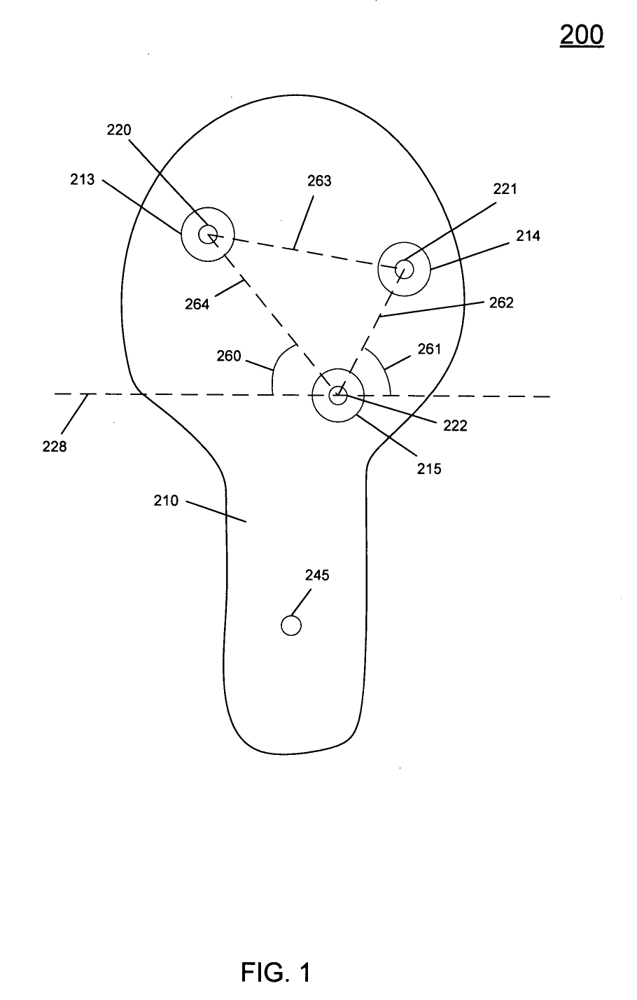

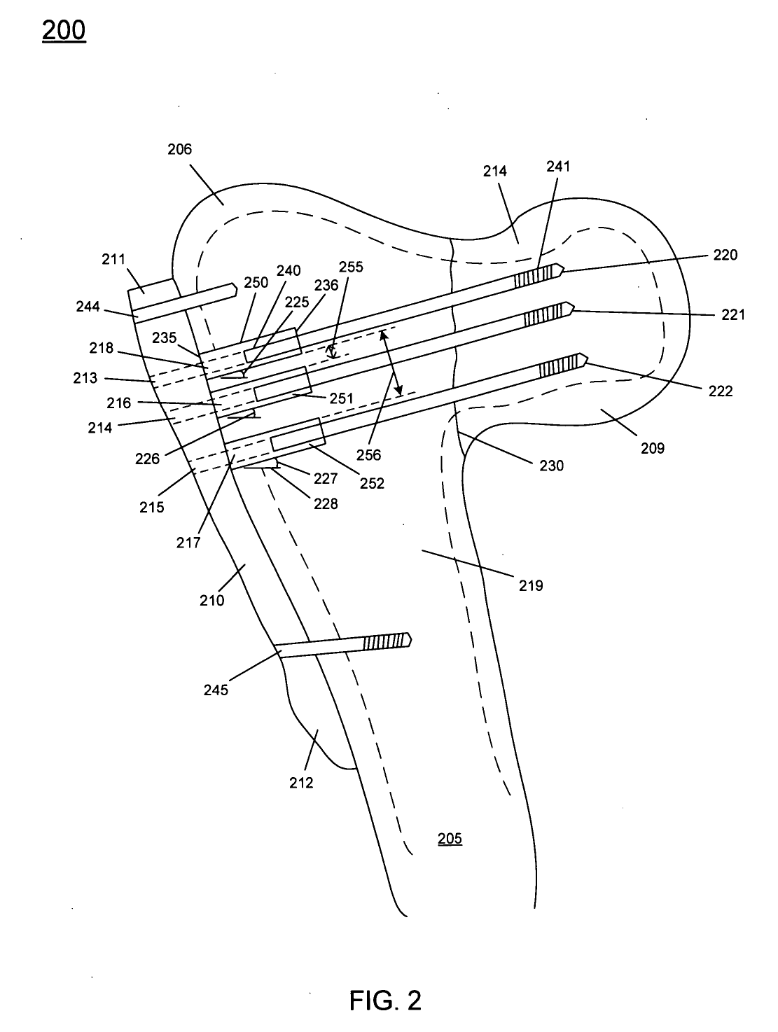

FIG. 1 and FIG. 2 are directed to one embodiment of the invention device 200. A base plate 210 may be attached to the femur. The base plate 210 may include an upper proximal region 211 and a lower distal region 212. The lower region 212 may couple to the bone 205 using screw 245 or screws. For example, an additional screw may be located above or below screw 245 in one embodiment of the invention. The upper region 211 may couple to the bone 205 using screw 244 or screws. The upper region 211 may include a hole 213 or void oriented at a predetermined angle 225 in relation to the horizontal plane and vertical axis of the plate 210, where the vertical axis is orthogonal to the horizontal plane. The upper region 211 may also include a second hole 214 or void oriented at another predetermined angle 226 and a third hole 215 or void oriented at a third predetermined angle 227.

A first barrel 250 may couple to the base plate 210. The first barrel 250 may include a circular cross-section, but other cross-sections (e.g., ovular) are also possible. In one embodiment of the invention, the barrel 250 may have a first open end 236 and a first closed end 235. The first barrel 250 may couple to the first hole 213 in the base plate 210 and to a first screw 220. For example, the first screw 220 may slide within hole 218 that is in barrel 250. The ability of the screw 220 to postoperatively slide within the hole 218 may result in better fixation when, for example, the bone 205 collapses or morphs postoperatively. The ability of the screw 220 to slide may also promote proper loading during, for example, walking or when the patient moves from a sitting to a standing position. A second barrel 251 may include a circular cross-section, or other cross-section, an open end, and a closed end. The second barrel 251 may couple to a hole 214 in the base 210 and to a second screw 221. Screw 221 may slide within hole 216 within barrel 251. A third barrel 252 may include a circular cross-section, or other cross-section, an open end, and a closed end. The third barrel 252 may couple to a hole 215 and to a third screw 222. Screw 222 may slide within hole 217 in barrel 252. In other embodiments of the invention, such as those described below, the barrel may include only open ends without any closed ends.

Again regarding FIGS. 1 and 2, in one embodiment of the invention the first barrel 250 may be formed or located a predetermined distance 255 from the second barrel 251 and a predetermined distance 256 from the third barrel 252. The interbarrel distances 255, 256, as well as the distance between the second barrel 251 and third barrel 252, may collectively reduce angular instability of a patient's fracture. For example, providing three fixation points appropriately spaced apart from one another may better resist rotation about the fracture 230 than, for example, a single sliding screw or two sliding screws.

In one embodiment of the invention, angles 225, 226, and 227 may be substantially the same. For example only, each angle may be between 25 degrees and 55 degrees (e.g., 30 degrees) above a horizontal plane 228 or 115 to 145 degrees from the vertical axis of the plate 210. However, broader angle ranges are included within the scope of the invention. When each angle 225, 226, 227 is substantially the same, the screws 220, 221, 222 may more easily slide within holes 218, 216, 217. However, in one embodiment of the invention each or some of the angles 225, 226, and 227 may be different. For example, in one embodiment of the invention angle 225 may be more displaced from a horizontal plane 228 (e.g., 45 degrees) than angle 227 (e.g., 25 degrees). These differing angles may help accommodate patients of differing sizes. For example, as angle 225 increasingly deflects from the horizontal plane 228 the spread between screws 220 and 222 increases, which may benefit a larger patient.

In one embodiment of the invention, various inserts (not illustrated) may be used to adjust angles 225, 226, and 227. For example, an insert (e.g., plug) may have external threads that screw into internal threads in base plate hole 213. The insert may be keyed so that its insertion position and final position in hole 213 is predetermined. The insert, in its final position, may reside inside the base plate 210 within hole 213. The insert may have a receiving hole for receiving barrel 250. The receiving hole may be offset from the horizontal plane at a predetermined angle that differs from the angle offset directly associated with hole 213. Thus, differing inserts may provide flexibility to a base plate that has holes (e.g., 213) set at predetermined angles. For example, hole 213 may be offset from the horizontal plane by 30 degrees. However, an insert with an additional 5 degrees of deflection may couple to hole 213 to create a 35 degree deflection. Multiple inserts with the same degree of offset may be used to keep all barrels/screws at the same angle. However, inserts with varying degrees of offset may be used in an embodiment where multiples screws should be placed at varying angles. In addition, in an embodiment of the invention, an insert may merely couple to hole 213 and reside outside of the base plate 210 (instead of inside hole 213 as described above). For example, the insert may have a neck with external threads that couple to internal threads in hole 213. In one embodiment of the invention, anther plate 210 is one of a set of plates that includes individual plates with holes oriented at angles different from that of plate 210. The surgeon may select from the various base plates according to the dimensions of the patient's hip.

In one embodiment of the invention, barrel 252 may slidably couple to screw 222. The surgeon may place screw 222 adjacent or near the femoral neck 207 inferior cortex 209. The screw 222 may indirectly couple to the inferior cortex 209 via cancellous bone. In other words, some marrow or less dense bone (e.g., cancellous bone) may be located between screw 222 and the inferior cortex 209. However, the screw 222 may directly contact the inferior cortex 209 (i.e., no cancellous bone between the screw and cortex) in some embodiments of the invention. In other words, when the screw is located “adjacent” to the cortex, the screw may be located functionally or efficaciously proximate (i.e., near) or directly on the hard cortex. Consequently, the cortex may provide stability to the adjacent screw as, for example, bone collapses post operatively or during certain loading events (e.g., a patient standing up). In contrast, if the screw 222 is not placed adjacent (i.e., not functionally near/on the cortex, as is the case when a “center-center” position, known to those of ordinary skill in the art, is used) to the cortex, the surrounding cancellous bone may be unable to provide adequate stability.

In some embodiments of the invention, an additional screw such as, for example, screw 221 may couple, directly or indirectly, to the posterior cortex 214. Screw 220 may also couple, directly or indirectly, with cortex (e.g., anterior cortex). For example, screws 220 and 221 may be positioned by the surgeon adjacent or near cortex tissue. Again, locating one or more screws 220, 221, 222 at or near the cortex provides stability due to the proximity of the screws to the dense cortex, as compared to softer inner bone. The additional stability may result in greater angular stability for the fracture 230. While none of the screws must be on the cortex to ensure angular stability, placing one or more of the screws adjacent, near, or on (i.e., directly coupled) the cortex may better ensure angular stability.

To facilitate placement of one or more of the screws adjacent or on the cortex, the barrels 250, 251, 252 (as described above), and consequently the holes 213, 214, 215 may be spaced predetermined distances from one another. For example, as seen in FIG. 1, the center of hole 214 may be spaced a distance 262 (e.g., 13-15 mm) from the center of hole 215. The center of hole 215 may be spaced a distance 264 (e.g., 13-15 mm) from the center of hole 213. The center of hole 213 may be spaced a distance 263 (e.g., 13-15 mm) from the center of hole 214. Hole 214 may be oriented at an angle 261 of, for example only, approximately 30-90 degrees (e.g., 60 degrees) above the horizontal plane 228 in relation to hole 215. Hole 213 may be oriented at an angle 260 of, for example only, approximately 30-90 degrees (e.g., 80 degrees) above the horizontal plane 228 in relation to hole 215. In an embodiment of the invention, holes 213, 214, 215 may be anteverted 0-30 degrees (e.g., 10 degrees). In one embodiment of the invention, holes 213, 214, 215 may act as drilling guides when drilling holes in the bone, placing guide wires in the bone holes, and/or placing the screws into the bone. Thus, the screws, when placed along the guide wires, are then located in a specifically spaced and angled pattern or orientation with, in one embodiment of the invention, one or more of the screws located on or adjacent to the bone cortex. The holes and barrels need not be placed symmetrically about any axis. For example, barrels 250, 251 are not necessarily positioned symmetrically about a central vertical axis of device 200. Instead, the barrels 250, 251, 252 may be positioned to promote specific placements of the screws (e.g., alignment of screws to bone cortex).

In one embodiment of the invention, barrels 250, 251, 252 each include an inner diameter of less than about 8 mm (e.g., 6.5 mm). The upper proximal end 211 of the base 210 may include a horizontal diameter or breadth greater than 20 mm (e.g., 28 mm) and a horizontal thickness of approximately 7 mm. The base lower distal end 212 may include a horizontal diameter less than 12 mm (e.g., 10 mm) and a horizontal thickness of approximately 5 mm. The screws 220, 221, 222 may include a maximum outer diameter necessary to properly couple to the inside of the barrel, e.g., approximately 6.5 mm, so the screws 220, 221, 222 may slide within the barrels 250, 251, 252. In some embodiments of the invention, some or all of the screws, and corresponding barrels, may have different diameters. For example, screw 222 (and barrel 252) may have a larger diameter to support a heightened load experienced in the inferior cortex region 209 of the bone 205. Furthermore, using smaller screws in the superior femoral head may help prevent avascular necrosis. Specifically, using smaller screws in the superior femoral head or more superior regions may avoid, for example, damaging the medial femoral circumflex artery and/or the retinacular vessel while distributing heavy loads to a larger diameter screw/screws located, for example, in the inferior femoral head. Placing a large diameter screw near the superior femoral head may lead to avascular necrosis.

In one embodiment of the invention, barrels 250, 251, 252 each include an open end to receive screws 220, 221, 222 and a closed end to prevent the screws from “backing out” of the bone post operatively. As bone settles post operatively, screws may begin to exit the bone and, for example, exit holes 213, 214, 215 if not prevented from doing so. As the screws exit holes 213, 214, 215 they may begin to irritate surrounding soft tissue. However, use of a barrel with a closed end 235 (or analogous cap, plug, etc.) will preclude such movement beyond a predetermined range of motion. Such a desired range of motion understandably stops short of allowing the screw to irritate soft tissue as described above.

In one embodiment of the invention the barrels are conduits with two open ends. With barrels of substantial lengths, the sliding and settling of the screws should be of a limited nature such that the pins or screws would not back out of the barrels. The conduits or barrels are used to guide and slidably receive screws 220, 221, 222. In an embodiment of the invention, the screws may have, for example, a projection or projections (not shown) to “catch” the barrels or the base itself if the screw moves beyond a desired sliding range. As another example, plugs may be inserted (e.g., screwed) into the base with the plug limiting the sliding range. The barrels may be permanently (e.g., welded or formed) or removably (e.g., screwed) coupled to the base. The barrels may directly or indirectly couple to the base. For example, as stated above, the barrels may couple to the base 210 via inserts of differing angles.

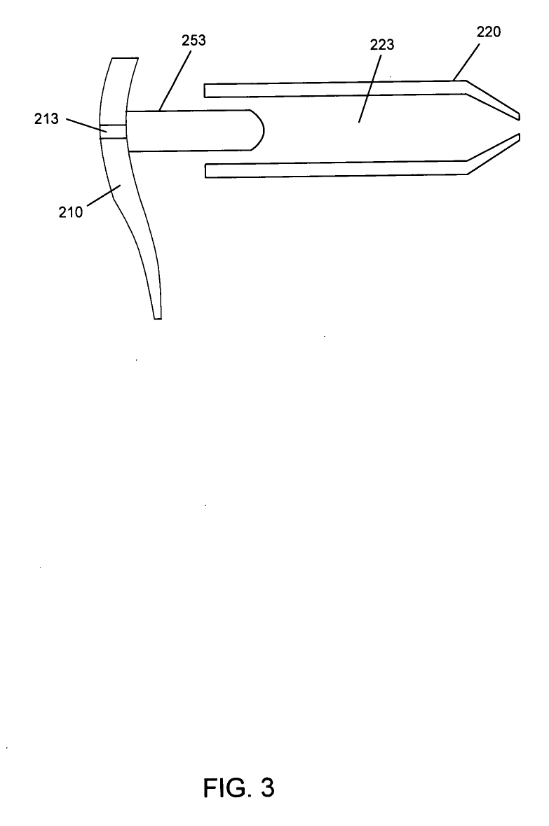

FIG. 3 includes a side cross-sectional view of a device in one embodiment of the invention. In this embodiment of the invention, the base 210 may couple, permanently or removably, to multiple projections such as, for example, columnar projection 253. For clarity, only one such projection is illustrated. In one embodiment of the invention, the plate hole 213, without projection 253 attached, may be used for guide wire placement. The plate 210 may be removed, a hole may be drilled, and screw 220 may then be placed into the bone over the guide wire. The plate 210, with projection 253 attached, may then be replaced with the projection 253 slidably received in channel 223 of the screw 220.

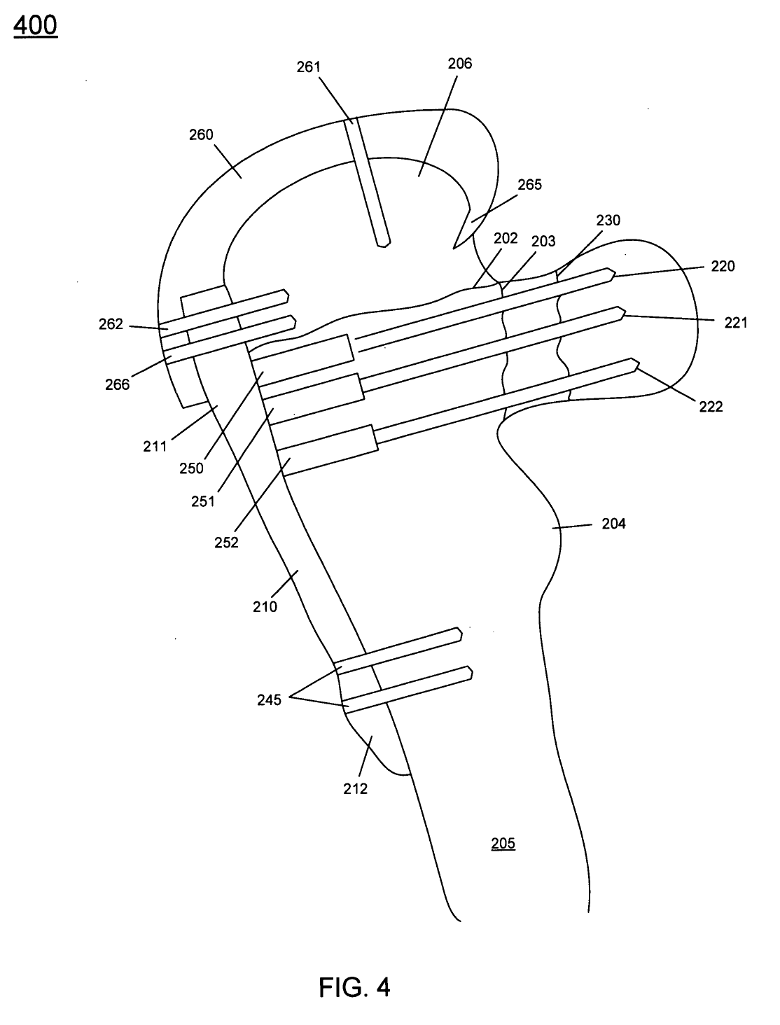

FIG. 4 includes one embodiment of the invention. A trochanter brace 260 couples to the upper plate region 211 and to the greater trochanter 206. A screw 261 or screws may couple an upper portion of the brace 260 to the bone 205 while another screw or screws 262, 266 fixate the lower brace to the bone 205 and to the upper plate region 211. The brace 260 may include a hook portion 265 to further facilitate fixation. As described above, the brace 260 may be removable from the base 210 (e.g., using, screws, dowels, latches, lugs). However, the brace 260 may also be permanently coupled to the base (e.g., using welds, common mold). The lower plate region 212 may couple to the bone 205 using screws 245. The brace 260 may stabilize the bone 205 when, for example, there is an intertrochanteric fracture 203 between the greater trochanter 206 and lesser trochanter 204, a fracture 202 in the greater trochanter, and/or a femoral neck fracture 230.

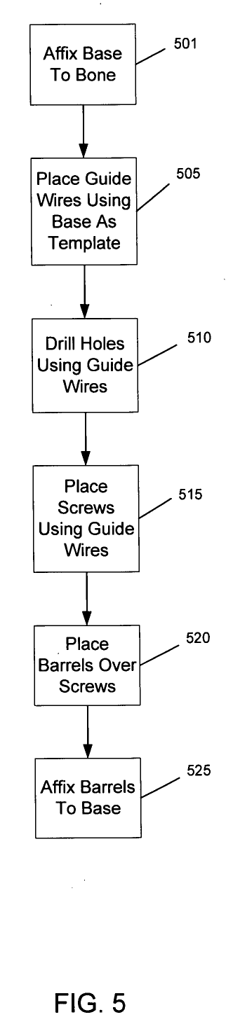

FIG. 5 includes a method in one embodiment of the invention. In block 501, a base plate 210 is affixed to the bone (e.g., femur) using a screw 245 (or screws) in the lower plate region 212. In block 505, the base holes 213, 214, 215 may be used as a template for placing the guide wires into the bone 205. As stated above, the holes 213, 214, 215 may be oriented at predetermined angles and distances to one another. This may facilitate eventually placing screws in, on, or near certain anatomical regions (e.g., inferior cortex) and thereby increasing angular stability.

In block 510, the base plate may be temporarily removed and a step drill may be used to drill holes over the guide wires. The step drill may include a drill bit of graduating diameters. For instance, the smallest diameter may form a pilot hole for the threaded portion of the screws 220, 221, 222. The next larger diameter may be approximately equal to any non-threaded portion of the screws 220, 221, 222. The next larger diameter may approximate the outer diameter of the barrels 250, 251, 252. In an embodiment of the invention, the base plate may be configured so that it need not be removed before using the step drill as described above. For example, enlarged holes 213, 214, 215 may be used to accommodate the drill. In an embodiment of the invention, various inserts may couple to the holes 213, 214, 215 to provide different diameters to the holes. For example, an insert with a smaller hole may be used for guide wire placement and an insert (or no insert) may be used for placing the barrels in the bone.

In block 515, the screws may be placed over the guide wires and into the holes drilled in block 510. As each screw is placed in the bone, torque is generated and rotational forces are applied to the bone. However, using a plurality of screws allows the surgeon to use screws of smaller diameters that, collectively, still manage the loads applied to the femoral neck. Placing screws with smaller diameters generates less torque per screw insertion. Furthermore, the initial placement of the multiple guide wires into the bone may stabilize the fracture and offset the rotational forces generated during screw placement.

In block 520, barrels are placed over the screws. In block 525, if the barrels are not permanently affixed to the base plate, they may be affixed to the base plate at this time. The barrels may have, for example, external threads that mate with internal threads in the base holes (e.g., 213, 214, 215). In another embodiment of the invention, the barrels are first placed over the guide wires and then the screws may be placed over the guide wires and into the holes drilled in block 510.

In one embodiment of the invention, if the barrels are permanently affixed to a plate, the plate with permanently affixed barrels may be affixed to the bone with the barrels being inserted into the bone. This base with permanently affixed barrels may be used in conjunction with, for example, an initial base that serves as a template for placing multiple screws in a predetermined pattern. In one embodiment of the invention, plugs may be used to seal one end of barrels having two opposed open ends. The plugs may have external threading that couples to internal threading in the barrel.

In one embodiment of the invention, the base of the implant is located substantially within a femoral intramedullary canal. The implant may function similarly to one or more of the above embodiments, albeit the base for the implant would be located in the intramedullary canal and not on the exterior of the femoral shaft. In one embodiment of the invention, the surgical technique used to insert the intramedullary implant may include the following steps. First, a 2 cm surgical incision may be placed proximal to the greater trochanter. Next, a channel would be surgically created from the tip of the greater trochanter into the medullary canal of the femur using, for example, an awl or mechanical drill. Next, the intramedullary device would be inserted into the medullary canal of the femur. Afterwards, a guiding device coupled to the intramedullary implant would be used to guide pins, drills, or screws through small incisions below the greater trochanter through the lateral cortex of the femur, through the intramedullary implant, and finally into the bone of the femoral head. As described above, there may be three or more holes in the implant to orient screws and the like so they receive support from adjacent cortex tissue in the femoral neck. The holes in the implant may be sized to receive sliding barrels that couple to sliding screws as described above. In one embodiment of the invention, the lower or distal portion of the intramedullary implant may have a 12 mm diameter while the upper or proximal portion has a 16 mm diameter. The implant shaft may include, for example, a customary four degree bend angling the upper portion of the implant away from the femoral neck, as those of ordinary skill in the art will appreciate. The screw to be located adjacent or near the femoral neck inferior cortex, as described above, may be positioned at an angle of, for example, 125 degrees from the lower or distal implant shaft.

Thus, in one embodiment of the invention, a femoral neck fracture fixation device 200 may include a base 210 having an upper end 211, lower end 212, inside surface, and outside surface; at least one bone fixation element 245 coupled to the lower end of the base component that fixes the inside surface of the base component 210 to the femoral bone 205; a first screw 220 coupled to the upper end 211 of the base component (e.g., via barrel 250) and extending into the femoral bone at an angle of eighty degrees (80°) to one hundred sixty degrees (160°) relative to the longitudinal axis (perpendicular to horizontal plane 228) of the base component; and at least a second screw 222 coupled to the upper end of the base component (e.g., via barrel 251) and extending into the femoral bone at an angle of eighty degrees (80°) to one hundred sixty degrees (160°) relative to the longitudinal axis of the base component. The fixation element can be a screw or multiple screws. Further, the device may include guide wires that penetrate the femoral bone prior to coupling the screws into place.

In various embodiments of the invention, the first screw and at least a second screw can be cannulated screws, compression screws, and the like. As noted above, the base component 210 can include a first receiving barrel 250 and at least a second receiving barrel 252 at the upper end 211 where the first screw 220 compressively couples to the first receiving barrel 250 and the at least a second screw 222 compressively couples to the at least a second receiving barrel 252. The compressively coupling screws may be compression screws. In at least one embodiment of the invention, the device includes three receiving barrels 250, 251, 252 at the upper end 211 of the base component and three screws 220, 221, 222 compressively coupled to the receiving barrels 250, 251, 252.

Alternatively, the device may include two, three, four, or more screws coupled to the upper end of the base component where each screw extends into the femoral bone at an angle of eighty degrees (80°) to one hundred sixty degrees (160°) relative to the longitudinal axis of the base component.

Although exemplary embodiments of the invention have been disclosed, it will be apparent to those skilled in the art that various changes and modifications can be made which will achieve some of the advantages of the invention without departing from the spirit and scope of the invention. For example, it will be obvious to those reasonably skilled in the art that, although the description was primarily directed to particular devices, other devices could be used in the same manner as that described. Other aspects, such as the specific methods utilized to achieve a particular function, as well as other modifications to the inventive concept are intended to be covered by the appended claims. For example, while one embodiment concerns stabilizing a femoral neck fracture of a human, other embodiments of the invention may be utilized for other animals including dogs.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. A femoral neck fracture fixation apparatus comprising:
a base plate including a vertical axis, a base plate upper proximal end and a base plate lower distal end, the base plate upper proximal end including a first hole oriented at a first predetermined angle from the vertical axis, a second hole oriented at a second predetermined angle from the vertical axis, and a third hole oriented at a third predetermined angle from the vertical axis;
a first barrel including a first open end and a second open end, the first barrel to couple to the first hole and to slidably couple to a first screw, the first screw to traverse and stabilize a femoral neck fracture;
a second barrel including a third open end and a fourth open end, the second barrel to couple to the second hole and to slidably couple to a second screw, the second screw to traverse and stabilize the femoral neck fracture; and
a third barrel including a fifth open end and a sixth open end, the third barrel to couple to the third hole and to slidably couple to a third screw, the third screw to traverse and stabilize a femoral neck fracture;
wherein the first barrel is a first predetermined distance from the second barrel and a second predetermined distance from the third barrel, the first predetermined distance and the second predetermined distance to collectively reduce angular instability of the femoral neck fracture.

2. The femoral neck fracture fixation apparatus of claim 1, wherein the first predetermined angle, the second predetermined angle, and the third predetermined angle are all substantially the same angle.

3. The femoral neck fracture fixation apparatus of claim 1, wherein the first predetermined angle is between 115 degrees and 145 degrees from the vertical axis.

4. The femoral neck fracture fixation apparatus of claim 1, wherein the first hole is spaced from the second hole to position the first screw adjacent to a femoral neck inferior cortex and to position the second screw in the femoral head.

5. The femoral neck fracture fixation apparatus of claim 4, wherein the first hole is spaced from the second hole to position the first screw adjacent to the femoral neck inferior cortex and to position the second screw adjacent to the femoral neck posterior cortex.

6. The femoral neck fracture fixation apparatus of claim 5, wherein the first hole is spaced from the third hole to position the first screw adjacent to the femoral neck inferior cortex and to position the third screw adjacent to the femoral neck anterior cortex.

7. The femoral neck fracture fixation apparatus of claim 5, wherein the first hole is spaced from the second hole to position the first screw no more than 5 mm from the femoral neck inferior cortex and to position the second screw no more than 5 mm from the femoral neck posterior cortex.

8. The femoral neck fracture fixation apparatus of claim 1, further comprising a trochanter brace that includes a trochanter brace upper proximal end and a trochanter brace lower distal end, the trochanter brace lower distal end to couple to the base plate upper proximal end and the trochanter brace upper proximal end to removably couple to a trochanter.

9. The femoral neck fracture fixation apparatus of claim 1, wherein the base plate lower distal end is to be located substantially within a femoral intramedullary canal.

10. An femoral neck fracture fixation apparatus comprising:
a base including a base upper proximal end and a base lower distal end;
a first conduit to couple to the base and to slidably receive a first screw that is to traverse a femoral neck fracture; and
a second conduit to couple to the base and to slidably receive a second screw that is to traverse the femoral neck fracture;
wherein the first conduit is spaced from the second conduit to locate the first screw adjacent to a femoral neck inferior cortex and to locate the second screw in the femoral neck thereby reducing angular instability of the femoral neck fracture.

11. The apparatus of claim 10, wherein the base lower distal end is to be located substantially within a femoral intramedullary canal.

12. The apparatus of claim 10, further comprising a third conduit to couple to the base and to slidably receive a third screw that is to traverse the femoral neck fracture;

13. A surgical method comprising:
locating a first screw across a femoral neck fracture and adjacent to the cortex of the femoral neck at a first predetermined angle;
locating a second screw across the femoral neck fracture and in the femoral head at a second predetermined angle and at a first predetermined distance from the first screw;
slidably coupling the first screw to a first conduit;
slidably coupling the second screw to a second conduit;
coupling a base member to the femur, the first conduit, and the second conduit; and
dynamically loading the femur and sliding the first screw within the first conduit and sliding the second screw within the second conduit.

14. The method of claim 13, further comprising sliding the first screw within a first range during dynamic loading of the femur, the first range extending between the base member and an open end of the first conduit.

15. The method of claim 13, further comprising locating the first screw no more than 5 mm from the femoral neck inferior cortex and locating the second screw no more than 5 mm from the femoral neck posterior cortex.

16. The method of claim 13, further comprising directly connecting the first screw to the cortex of the femoral neck.

17. The method of claim 13, further comprising coupling a trochanter brace to a trochanter and to the base member.

18. The method of claim 13, further comprising:
locating the first screw adjacent to the inferior cortex of the femoral neck; and
locating the second screw adjacent to the posterior cortex of the femoral neck.

19. The method of claim 13, further comprising:
coupling the first screw to a first guide wire; and
coupling the second screw to a second guide wire.

20. The method of claim 19, further comprising an additional base member that includes an additional vertical axis, an additional base member upper proximal end and an additional base member lower distal end, the additional base member upper proximal end including a fourth hole oriented at a fourth predetermined angle from the additional vertical axis, a fifth hole oriented at a fifth predetermined angle from the additional vertical axis, and a sixth hole oriented at a sixth predetermined angle from the additional vertical axis; wherein the first barrel is to couple to the fourth hole, the second barrel is to couple to the fifth hole, and the third barrel is to couple to the sixth hole, the fourth predetermined angle being greater than the first predetermined angle.
